(19) United States
(12) Patent Application Publication
(10) Pub. No.: US 2020/0300771 A1
(43) Pub. Date: Sep. 24, 2020

(54) NITROGEN MOLECULAR SENSOR FOR DETECTING NITROGEN CONTENT IN PLANT AND USE THEREOF

(71) Applicant: SEOUL NATIONAL UNIVERSITY R&DB FOUNDATION, Seoul (KR)

(72) Inventors: Ju-Kon KIM, Seoul (KR); Dong-Keun LEE, Gangwon-do (KR)

(21) Appl. No.: 16/649,370

(22) PCT Filed: Sep. 22, 2017
(86) PCT No.: PCT/KR2017/010460

§ 371 Date: Mar. 20, 2020

(51) Int. Cl. G01N 21/76; G01N 21/64; G01N 33/00; G01N 33/50

(52) U.S. Cl. CPC G01N 21/763; G01N 33/5097; G01N 33/0098; G01N 21/6486

(57) ABSTRACT

A nitrogen molecular sensor according to an embodiment of the present invention detects the nitrogen content in a plant. The nitrogen molecular sensor is manufactured by a novel method that includes the isolation of new nitrogen-sensitive genes. Transgenic rice plant containing the nitrogen sensor eventually may respond to nitrogen with high sensitivity. The biological nitrogen sensor can be applied to develop the crops improved nitrogen use efficiency through overcoming the current limitation of the phenotype characterization related to nitrogen metabolism in plants. As a result, it can be used as a core technology for isolating and analyzing industrially valuable genes involving in crop nitrogen use efficiency using a mutant pool harboring nitrogen sensor.

### TECHNICAL FIELD

The present invention relates to a nitrogen molecular sensor for detecting the nitrogen content in a plant, and a use thereof.

### BACKGROUND ART

Nitrogen is one of the essential inorganic nutrients in the plant, and it is the main component of nucleic acids, proteins, various types of cofactors, and secondary metabolites. In plants, nitrate is a strong signal which influences not only the metabolism of nitrogen and carbon but also the growth and development of organs.

During the last 50 years, the development of an effective irrigation system and fertilizer has met the increasing food demand in proportion to the exponential growth of the global human population. Accordingly, the global application of the fertilizer, including nitrogen, phosphate, and potassium, has been increased consistently to enhance food production. However, excessive application of fertilizer results in serious environmental problems, including eutrophication and greenhouse gas emissions, and also adverse effects on the agricultural economy. The most effective way to overcome those significant problems is to develop a method to enhance the crop yield using the minimal application of fertilizer. Therefore the demands for scientific study on such a technique are now more reliable than ever before. The essential requirement for this approach is obtaining core genetic resources by isolating and analyzing a nitrogen specific character. However, the characterization of a particular phenotype related to nitrogen metabolism has been limited so far, and it is believed that a noble approach for detecting internal nitrogen status in a plant is mainly required. Accordingly, a sensitive nitrogen biosensor is now developed.

Meanwhile, in Korean Patent Publication No. 2010-0007600, “Use of OsHXK5 gene as glucose sensor” is disclosed, and, in Korean Patent Publication No. 2012-0081270, “Method for controlling nitrogen assimilation and disease tolerance of plant using AtSIZ1 gene” is disclosed. However, no disclosure has been made regarding the nitrogen molecular sensor for detecting nitrogen content in a plant and a use thereof as described in the present invention.

### DETAILED DESCRIPTION OF THE INVENTION

### Technical Problems to be Solved

The present invention is devised under the circumstances described above. In the present invention, a biological nitrogen molecular sensor is manufactured by utilizing a nitrogen-sensitive transcriptional response of ALLANTOINASE (OsALN) and UREIDE PERMEASE 1 (Os UPS1) derived from rice (Oryza sativa). And it is confirmed that the nitrogen state in a plant can be quickly and sensitively measured in a non-disruptive manner using this biological nitrogen molecular sensor.

Specifically, a biological nitrogen molecular sensor, proALN::ALN-LUC2 and proUPS1::UPS1-LUC2, is prepared by utilizing the nitrogen sensitivity. The transgenic rice plant harboring proUPS1::UPS1-LUC2, which is obtained after transformation of rice (Oryza sativa), exhibits a strong luminescence activity in a nitrogen-sufficient condition, and it indicates that internal nitrogen content is sufficient in the plant. In addition, as the luminescence activity is low at a nitrogen-deficient condition, it indicates that internal nitrogen content is deficient in the plant. On the contrary, as the transgenic rice plant harboring proALN::ALN-LUC2 exhibits a weak luminescence activity in a nitrogen-rich state, it indicates that internal nitrogen content is sufficient in the plant. In addition, as the luminescence activity is high at a nitrogen-deficient condition, it indicates that internal nitrogen content is deficient in the plant.

Accordingly, the present invention is completed based on finding that a biological nitrogen molecular sensor can be manufactured by utilizing the nitrogen-sensitive transcriptional response of OsALN and OsUPS1 genes.

### Technical Means for Solving the Problems

In order to achieve the goals described above, the present invention provides a nitrogen molecular sensor for detecting nitrogen content in a plant characterized by comprising a plant transformed with an expression vector including ALN (ALLANTOINASE) gene derived from rice (Oryza sativa) and a gene encoding a luminescent protein or with an expression vector including UPS1 (UREIDE PERMEASE 1) gene derived from rice (Oryza sativa) and a gene encoding a luminescent protein.

The present invention further provides a method for measuring nitrogen content in a plant, including: (a) preparing a plant transformed with an expression vector including ALN (ALLANTOINASE) gene derived from rice (Oryza sativa) and a gene encoding a luminescent protein or with an expression vector including UPS1 (UREIDE PERMEASE 1) gene derived from rice (Oryza sativa) and a gene encoding a luminescent protein; and (b) cultivating a transgenic plant of the above (a) and measuring the luminescence intensity of the transgenic plant.

The present invention further provides a composition for measuring nitrogen content in a plant comprising of an expression vector including ALN (ALLANTOINASE) gene derived from rice (Oryza sativa) and a gene encoding a luminescent protein or an expression vector including UPS1 (UREIDE PERMEASE 1) gene derived from rice (Oryza sativa) and a gene encoding a luminescent protein as an effective component.

### Advantageous Effect of the Invention

The present invention relates to a nitrogen molecular sensor for detecting the nitrogen content in a plant and use thereof. In the present invention, a nitrogen molecular sensor is manufactured by an original method which has not been studied thus far. The original method includes the isolation of new nitrogen-sensitive genes, the manufacture of a nitrogen biomolecular sensor, and the results in which a transgenic rice plant containing the nitrogen sensor eventually responds to nitrogen with high sensitivity. Ultimately, the biological nitrogen sensor, which is the product of the present invention, can be applied to develop the crops improved nitrogen use efficiency through overcoming the current limitation of the phenotype characterization related to nitrogen metabolism in a plant. As a result, it can be used as a core technology for isolating and analyzing industrially valuable genes involving in crop nitrogen use efficiency using a mutant pool harboring the nitrogen sensor.

### BRIEF DESCRIPTION OF THE DRAWINGS

FIG. 1 shows the results that allantoin functions as a nitrogen source in rice. (A) Allantoin degradation pathway. In the allantoin degradation pathway, allantoin is converted to glyoxylate to generate 4 NH₃ eventually. In the figure, the abbreviations are as follows: ALN, ALLANTOINASE; AAH, ALLANTOATE AMIDOHYDROLASE; UGlyAH, UREIDOGLYCINE AMINOHYDROLASE; UAH, UREIDOGLYCOLATE AMINOHYDROLASE. In the figure, (B) phenotype of the rice (9-day-old) which has been cultivated in a growth medium containing allantoin, as a sole nitrogen source, at various concentrations, (C) height of the plant (9-day-old), and (D) SPAD value of the plant (16-day-old) are shown. The values represent the mean±SD of two tests. Each test was carried out with 20 plants at every concentration of allantoin.

FIG. 2 shows the results indicating that levels of allantoin metabolites in ricc are sensitivity changed depending on the state of N. (A) is a diagram representing the test of N depletion and N re-application. Dongjin wild type was cultivated for 21 days in soil with a nitrogen source and Yoshida solution for hydroponic cultivation. Young plants were grown for 10 more days at a condition of N depletion using Yoshida solution having no nitrogen source. After the N depletion condition, 2.8 mM NH₄NO₃ (final concentration) was supplied to create a condition of N re-application. Using the shoot and root samples, allantoin (B), allantoic acid (C), and ureidoglycolate (D) were collected from the samples under N depletion and re-application condition, and then their quantitative values were obtained. The given values represent the mean±S.D. of two biological and technical repetition groups.

FIG. 3 shows the expression pattern of OsALN and OsUPS1, which rapidly changes depending on a different status of nitrogen. Transcription level of OsALN (A,E), OsAAH (B,F), Os UGlyAH (C,G), and Os UPS1 (D,H) were represented by qRT-PCR analysis, and expression data in shoot (A-D) and root (E-H) tissues were also represented. The expression of OsUBI1 was used as an internal control. The given values represent the mean±SD of two biological and technical repetition groups. * indicates a significant difference within 95% confidence interval obtained from Students' t-test.

FIG. 4 shows the profiles of proALN::ALN-LUC2 and proUPS1::UPS1-LUC2 transgenic rice plants. Vector maps of proALN::ALN-LUC2 (A) and proUPS1::UPS1-LUC2 (B) are represented. The phenotypes of T₀ rice with proALN::ALN-LUC2 (C) and proUPS1::UPS1-LUC2 (D) are shown. The results of TaqMan q-PCR for nos terminator of proALN::ALN-LUC2 (E) and proUPS1::UPS1-LUC2 (F) of T₀ rice are shown. The oligonucleotide probe targeted at TUBULIN 1 of rice (OsTub1, Os11g0247300) was designed and used for qPCR standardization as the internal control group. The transgenic plants inserted with single-copy homozygous T-DNA, which has been previously isolated, was used as a positive control group (C). Copy number of the genes that are transgenically introduced to T₀ proALN::ALN-LUC2 (G) and proUPS1::UPS1-LUC2 (H) rice is represented.

FIG. 5 shows the results that the transcriptional regulation of proALN::ALN-LUC2 and proUPS1::UPS1-LUC2 is closely related with the expression pattern of an endogenous gene regulated by the status of nitrogen. The results of qRT-PCR analysis of pro UPS1::UPS1-LUC2 (A) and proALN::ALN-LUC2 (B) from shoots and roots during the nitrogen depletion and repletion test are represented. UPS1-LUC2 and ALN-LUC2 represent a transgenically introduced gene, each derived from proUPS1::UPS1-LUC2 and proALN::ALN-LUC2, respectively. Expression of OsUBI1 was used as an internal control. The given values represent the mean±S.D. of two biological and technical repetition groups. * indicates a significant difference within 95% confidence interval obtained from Students' t-test.

FIG. 6 shows that the luminescence activity of proUPS1::UPS1-LUC2 is high at a condition of high N concentration. In the figure, (A) represents the image of NT and proUPS1::UPS1-LUC2 plants grown for 5 days in growth media with high levels of N sources, including 20 mM ammonium nitrate and 19 mM potassium nitrate (GM+N), or on growth media without N sources (GM−N). (B) represents the relative intensity of the luminescence signal of T₃ homozygous proUPS1::UPS1-LUC2 plants at the same condition as (A). (C) represents the image of NT and proUPS1::UPS1-LUC2 plants which have been grown for 5 days in GM-N, or the image of NT and proUPS1::UPS1-LUC2 plants which have been grown for 4 days in GM-N followed by further growing for 1 day after the addition of 100 mM ammonium nitrate. (D) represents the relative intensity of luminescence signal of T₃ homozygous proUPS1::UPS1-LUC2 plants at the same condition as (C). The given data represent mean±S.D. of five T₃ homozygous proUPS1::UPS1-LUC2 lines (n=10 for each transgenic plant). * indicates a significant difference within 95% confidence interval obtained from Students' t-test. NT grown in GM-N was employed as a control group for standardization. * indicates a significant difference within 95% confidence interval obtained from Students' t-test.

FIG. 7 shows that the luminescence activity of proALN::ALN-LUC2 is high at a condition with low N concentration. In the figure, (A) represents the image of NT and proALN::ALN-LUC2 plants grown for 5 days in GM+N or GM-N. (B) represents the relative intensity of luminescence signal of T₃ homozygous proALN::ALN-LUC2 plants at the same condition as (A). (C) represents the image of NT and proALN::ALN-LUC2 plants which have been grown for 5 days in GM-N, or the image of NT and proALN::ALN-LUC2 plants which have been grown for 4 days in GM-N followed by further growing for 1 day after the addition of 100 mM ammonium nitrate. (D) represents the relative intensity of luminescence signal of T₃ homozygous proALN::ALN-LUC2 plants in the same condition as (C). The given data represent mean±S.D. of five T₃ homozygous proALN::ALN-LUC2 lines (n=10 for each transgenic plant). * indicates a significant difference within 95% confidence interval obtained from Students' t-test. NT grown in GM-N was employed as a control group for standardization. * indicates a significant difference within 95% confidence interval obtained from Students' t-test.

FIG. 8 shows the N substrate specificity of proUPS1::UPS1-LUC2 and proALN::ALN-LUC2 plants. After growing the plants for 1 day with addition of 100 mM ammonium nitrate, ammonium sulfate, or potassium nitration followed by growing for 4 days in GM-N, i.e., growing for 5 days in total, the relative intensity of luminescence signal from T₃ homozygous proUPS1::UPS1-LUC2 (A) and proALN::ALN-LUC2 (B) plants was shown. The given data represent mean±S.D. of five T₃ homozygous lines of transgenic plants harboring the N sensor (n=10 for each transgenic plant).

FIG. 9 shows the nitrogen response range of proUPS1::UPS1-LUC2 and proALN::ALN-LUC2 plants. The relative intensity of luminescence signal in T₃ homozygous proUPS1::UPS1-LUC2 (A-C) and proALN::ALN-LUC2 (D-F) plants was shown. The plants was grown for 1 day with the addition of 0, 0.0001, 0.001, 0.01, 0.1, 1, 10, 100, or 1000 mM ammonium nitrate (A, D), ammonium sulfate (B, E), or potassium nitration followed by growing for 4 days in GM-N. The given data represent mean±S.D. of five T₃ homozygous lines of transgenic plants harboring the N sensor (n=10 for each transgenic plant).

### BEST MODE(S) FOR CARRYING OUT THE INVENTION

To achieve the goal, the present invention provides a nitrogen molecular sensor for detecting nitrogen content in a plant comprising a plant transformed with an expression vector including ALN (ALLANTOINASE) gene derived from rice (Oryza sativa) and a gene encoding a luminescent protein or transformed with an expression vector including UPS1 (UREIDE PERMEASE 1) gene derived from rice (Oryza sativa) and a gene encoding a luminescent protein.

In the nitrogen molecular sensor of the present invention, the plant transformed with an expression vector including ALN gene derived from Oryza sativa and a gene encoding a luminescent protein may exhibit the luminescence at a nitrogen-deficient condition. And the plant transformed with an expression vector including UPS1 gene derived from Oryza sativa and a gene encoding a luminescent protein may exhibit the luminescence at a nitrogen-sufficient condition.

In the nitrogen molecular sensor of the present invention, the nitrogen-deficient condition may be 0.1 mM or less concentration of nitrogen source. Moreover, the nitrogen-sufficient condition may be 1 mM or more concentration of nitrogen source, but it is not limited thereto.

In the nitrogen molecular sensor of the present invention, ALN gene may consist of the nucleotide sequence of SEQ ID NO: 1, and UPS1 gene may consist of the nucleotide sequence of SEQ ID NO: 2, but it is not limited thereto.

Homologs of the above sequences are also encompassed in the scope of the present invention. The homolog indicates a nucleotide sequence having functional characteristics that are similar to those of the nucleotide sequence of SEQ ID NO: 1 or SEQ ID NO: 2 despite a change in the nucleotide sequence. Specifically, each of ALN (ALLANTOINASE) gene and UPS1 (UREIDE PERMEASE 1) gene may include a nucleotide sequence having at least 70%, preferably at least 80%, more preferably at least 90%, and even more preferably at least 95% sequence homology with the nucleotide sequence of SEQ ID NO: 1 or SEQ ID NO: 2.

The “sequence homology %” for a certain polynucleotide is identified by an optimal alignment of a comparative region with two sequences. In this regard, a part of the polynucleotide in the comparative region may include an addition or a deletion (i.e., a gap) compared to a reference sequence (without any addition or deletion) after optimizing the alignment of the two sequences.

The term “vector” is used for indicating a DNA fragment(s), a nucleic acid to be delivered to the inside of a cell. Vector allows DNA replication and can be independently reproduced in a host cell. The “delivery system” is often interchangeably used with the term “vector”. The term “expression vector” means a recombinant DNA molecule comprising of a desired coding sequence and other appropriate nucleotide sequences that are essential for the expression of the operatively-linked coding sequence in a specific host organism. The promoter, enhancer, termination signal, and polyadenylation signal that can be used in eukaryotic cells are well known.

The expression vector preferably comprises at least one selective marker. The above selective marker is a nucleotide sequence having a property of being selected by a common chemical method, and examples thereof include all genes applicable for distinguishing transformed cells from non-transformed cells. Specific examples include a gene resistant to herbicide (e.g., glyphosate and phosphinothricin) and a gene resistant to antibiotics (e.g., kanamycin, G418, bleomycin, hygromycin, and chloramphenicol), but they are not limited thereto.

For the plant expression vector according to one embodiment of the present invention, the promoter may be CaMV 35S promoter, actin promoter, ubiquitin promoter, pEMU promoter, MAS promoter, or histone promoter, but not limited thereto.

As for the terminator, any conventional terminator can be used. Examples thereof include nopaline synthase (NOS), rice α-amylase RAmy1 A terminator, a phaseolin terminator, or a terminator for octopine gene of Agrobacterium tumefaciens but, they are not limited thereto.

In the nitrogen molecular sensor according to one embodiment of the present invention, the luminescent protein may be luciferase, GFP (green fluorescent protein), EGFP (enhanced green fluorescent protein), GFPuv (cycle 3 variant of GFP), EBFP (enhanced blue fluorescent protein), ECFP (enhanced cyan fluorescent protein), or YFP (yellow fluorescent protein), and preferably luciferase, but it is not limited thereto.

The present invention further provides a method for measuring nitrogen content in a plant, including:

(a) preparing a plant transformed with an expression vector including ALN (ALLANTOINASE) gene derived from rice (Oryza sativa) and a gene encoding a luminescent protein or an expression vector including UPS1 (UREIDE PERMEASE 1) gene derived from rice (Oryza sativa) and a gene encoding a luminescent protein; and

(b) cultivating a transgenic plant of the above (a) and measuring the luminescence intensity of the transgenic plant.

Plant transformation means any method by which DNA is delivered to a plant. Such a transformation method does not necessarily need a period for regeneration and (or) tissue culture. Transformation of plant species is now quite general for plant species including not only dicot plants but also monocot plants. In principle, any transformation method can be used for introducing a hybrid DNA of the present invention to appropriate progenitor cells. The method can be appropriately selected from a calcium/polyethylene glycol method for protoplasts (Krens, F. A. et al., 1982, Nature 296, 72-74), an electroporation method for protoplasts (Shillito R. D. et al., 1985 Bio/Technol. 3, 1099-1102), a microscopic injection method for plant components (Crossway A. et al., 1986, Mol. Gen. Genet. 202, 179-185), a (DNA or RNA-coated) particle bombardment method for various plant components (Klein T. M. et al., 1987, Nature 327, 70), or a (non-complete) viral infection method in Agrobacterium tumefaciens mediated gene transfer by plant invasion or transformation of fully ripened pollen or microspore, etc. A method preferred in the present invention includes Agrobacterium mediated DNA transfer. In particular, the so-called binary vector technique, as disclosed in EPA 120 516 and U.S. Pat. No. 4,940,838, can be preferably used for the present invention.

In the method according to one embodiment of the present invention, ALN gene may consist of the nucleotide sequence of SEQ ID NO: 1, and UPS1 gene may consist of the nucleotide sequence of SEQ ID NO: 2, but they are not limited thereto.

In the method according to one embodiment of the present invention, the luminescent protein may be luciferase, GFP (green fluorescent protein), EGFP (enhanced green fluorescent protein), GFPuv (cycle 3 variant of GFP), EBFP (enhanced blue fluorescent protein), ECFP (enhanced cyan fluorescent protein), or YFP (yellow fluorescent protein), and preferably luciferase, but it is not limited thereto.

The plant according to the present invention can be a monocot plant such as rice, barley, corn, wheat, rye, oat, meadow grass, fodder grass, millet, sugar cane, ryegrass, or orchard grass, or a dicot plant such as Arabidopsis thaliana, potato, eggplant, tobacco, pepper, tomato, burdock, crown daisy, lettuce, balloon flower, spinach, chard, yam, celery, carrot, water parsley, parsley, Chinese cabbage, cabbage, radish, watermelon, oriental melon, cucumber, zucchini, gourd, strawberry, soybean, mung bean, kidney bean, or sweet pea. Preferably, it can be rice, but it is not limited thereto.

The present invention still further provides an expression vector including ALN (ALLANTOINASE) gene derived from rice (Oryza sativa) and a gene encoding a luminescent protein because the plant transformed with an expression vector including ALN (ALLANTOINASE) gene and a gene encoding a luminescent protein may generate the luminescence at a nitrogen-deficient condition. Besides, the present invention provides an expression vector including UPS1 (UREIDE PERMEASE 1) gene derived from rice (Oryza sativa) and a gene encoding a luminescent protein for measuring nitrogen content in a plant because the plant transformed with an expression vector including UPS1 gene and a gene encoding a luminescent protein may generate the luminescence at a nitrogen-sufficient condition.

Hereinbelow, the present invention is explained in greater details in view of the Examples. However, it is evident that the following Examples are given only for exemplification of the present invention and don't mean the present invention is limited to the following Examples.

### EXAMPLES

### Example 1. Role of Allantoin as a Nitrogen Source in Rice

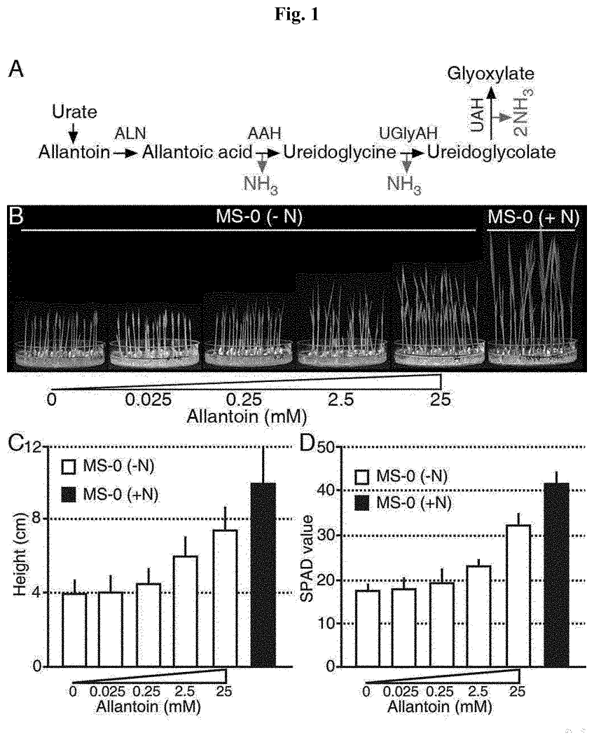

Allantoin is reported as a major nitrogen source of legumes. However, for common plants other than legumes, the biological role of allantoin has not been reported clearly. In particular, the likelihood of allantoin functioning as a nitrogen source is reported from Arabidopsis thaliana, but its role in a monocot plant, including rice, has not been reported. In the present invention, it is systemically determined that allantoin serves as a nitrogen source in rice (FIG. 1). It is shown that rice can grow and develop continuously by applying allantoin as a sole nitrogen source. In particular, the synthesis of chlorophylls in chloroplast occurs in a normal way. Therefore utilization of allantoin as a nitrogen source in rice is identified.

MS-O (-N) shown in FIG. 1B represents the growth of rice under a condition in which allantoin is supplied to a nutrition medium, which is free of any nitrogen source, as a sole nitrogen source with different concentrations. The results indicate that, once the allantoin reaches a certain level of mM stage, the growth and development is restored to the normal level like rice grown at MS-O (+N) condition containing 20 mM ammonium nitrate as a general nitrogen source. It is shown that the deficiency of nitrogen source is closely related to the growth of rice and density of chlorophylls—further, FIGS. 1C and 1D indicate that, when allantoin was added at mM level, the growth of rice and density of chlorophylls are similar to those of rice grown at normal conditions.

### Example 2. Change in Allantoin Metabolites According to the Increase and Decrease of Nitrogen Content in Rice

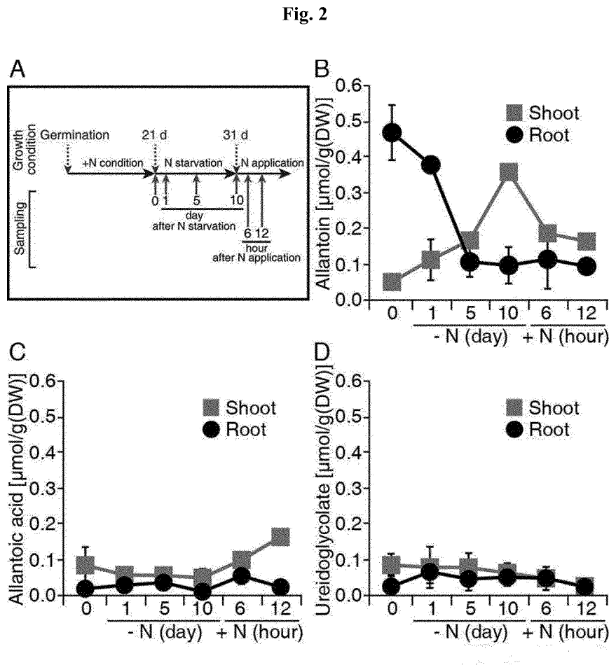

According to the presented Example 1, it was confirmed that allantoin is used as a nitrogen source in rice. For allantoin to be used as a nitrogen source, it is necessary to produce ammonia by subsequent degradation of allantoin. To understand this degradation process of allantoin for generating a nitrogen source, a change of allantoin metabolites was monitored after creating nitrogen-deficient condition (FIG. 2). It was found that, under a nitrogen-deficient condition, allantoin in roots is degraded and lost rapidly, while allantoin accumulates in the aboveground part (FIG. 2B). But allantoic acid (FIG. 2C) and ureidoglycolate (FIG. 2D), which are allantoin-derived subsequent metabolites in allantoin degradation pathway, show no change. Therefore it suggests that allantoin functions as a limiting factor for providing nitrogen source via allantoin degradation. On the other hand, when the nitrogen is fed again, it was observed that the allantoin metabolites in the aboveground part are restored to the original level. These results indicate that allantoin is used as a nitrogen source in rice.

### Example 3. Expression Pattern of Genes Involved in Allantoin Metabolism According to Increase and Decrease of Nitrogen Content in Rice

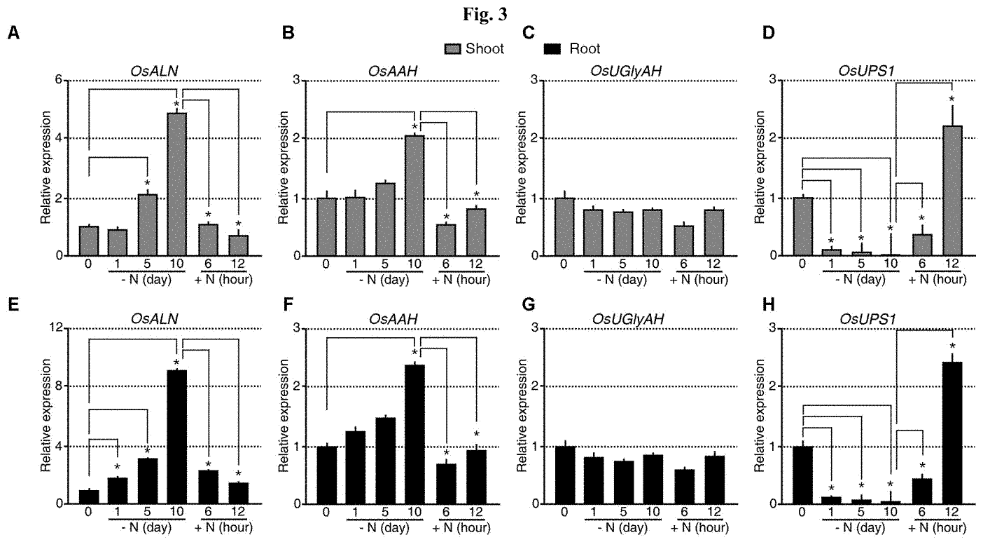

According to the above Examples 1 and 2, it was confirmed that allantoin is used as a nitrogen source in rice. To verify the evidence at a gene expression level, the expression pattern of the genes involved in allantoin degradation pathway was analyzed at a nitrogen-deficient or nitrogen re-application condition (FIG. 3). Under the nitrogen-deficient condition, expression of OsALN and OsAAH genes, which are the first gene for degrading allantoin, has been sensitively induced in rice, but the expression of OsALN and OsAAH genes was repressed again under nitrogen re-application condition. Based on these results, it was recognized that, under a nitrogen-deficient condition, allantoin is degraded to produce a nitrogen source in ammonia form, but it is not degraded under the nitrogen-sufficient condition. On the other hand, the expression level of Os UPS1, which is responsible for allantoin transportation, was decreased under the nitrogen-deficient condition but increased again under nitrogen re-application condition. These results suggest that allantoin transportation does not occur in nitrogen-deficient condition since the allantoin needs to be used as a nitrogen source. But allantoin is transferred by OsUPS1 in nitrogen sufficient condition. All of these experimental results (FIG. 1, FIG. 2 and FIG. 3) indicate that allantoin is utilized as a nitrogen source in rice.

### Example 4. Manufacture of Nitrogen Molecular Sensor

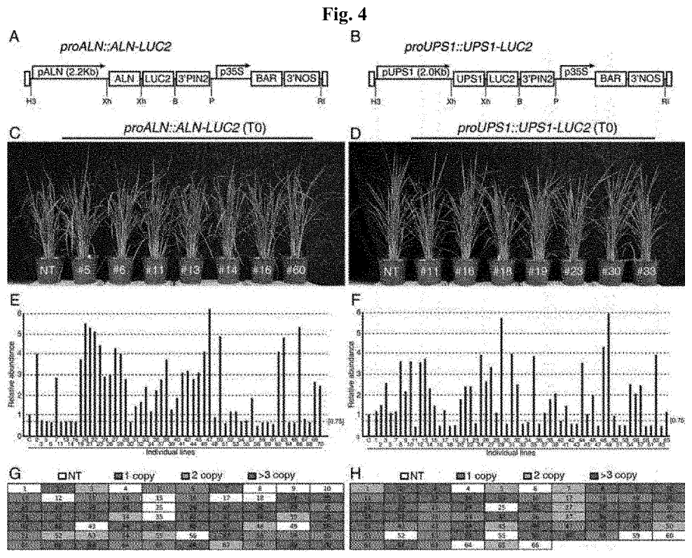

According to the above Example 3, it was found that the expression pattern of OsALN and Os UPS1 genes in rice showed an opposite pattern depending on the state of nitrogen in rice and their response sensitivity is very high. By utilizing this response sensitivity to nitrogen, a nitrogen molecular sensor was manufactured. With use of luciferase as a reporter gene, each gene was translationally fused to produce proALN::ALN-LUC2 and pro UPS1::UPS1-LUC2 followed by transformation of rice with them (FIG. 4). For each nitrogen molecular sensor construct, 66 to 70 transformants were generated, and transformants harboring a single copy of the nitrogen molecular sensor were isolated by using Taqman PCR. Isolated transformants were proliferated to form a homozygote, and, finally, five homozygous single-copy transformants were isolated for each nitrogen molecular sensor.

### Example 5. Transcriptional Response of Transformant Harboring Nitrogen Molecular Sensor to Nitrogen

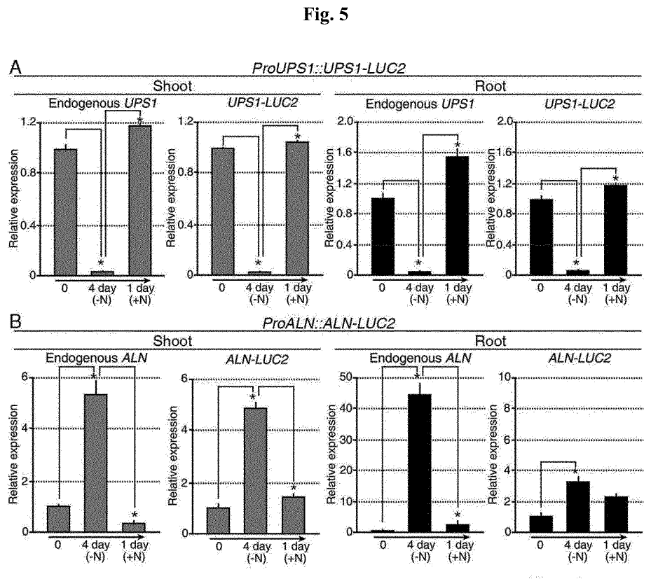

To determine the response of the transformant harboring nitrogen molecular sensor to nitrogen, a transcriptome of the transformants was isolated after the nitrogen depletion or nitrogen re-application, and then the expression pattern between endogenous OsALN and Os UPS1, and the nitrogen molecular sensor were compared and analyzed. Accordingly, it was found that each molecular sensor shows a very similar transcriptional pattern with endogenous OsALN and Os UPS1 response to nitrogen. Therefore the ability of the nitrogen molecular sensor for detecting nitrogen content in plant was confirmed (FIG. 5).

### Example 6. Luminescence Activity Responding to Nitrogen in Transformant Harboring Nitrogen Molecular Sensor

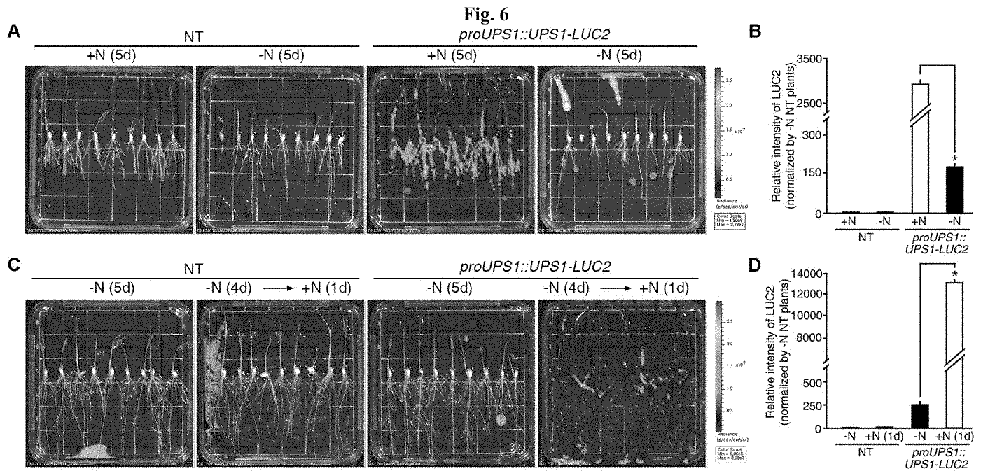

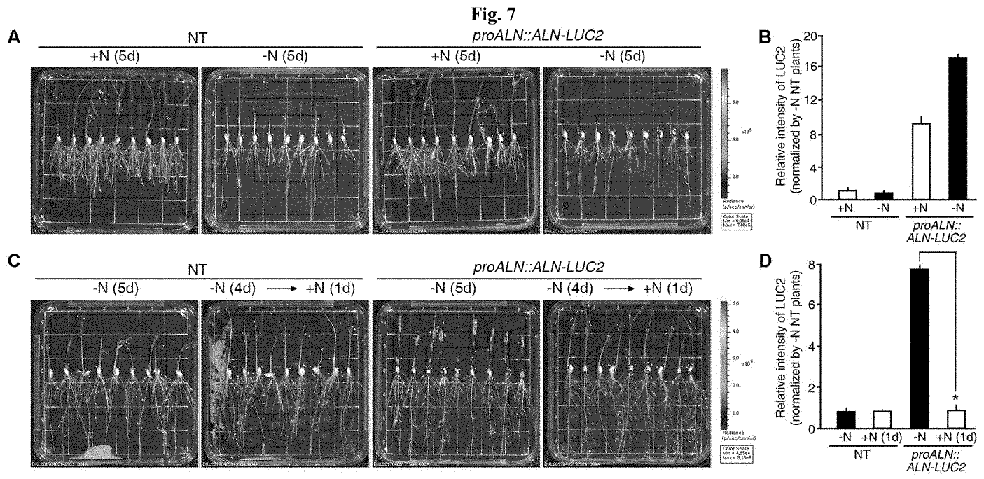

To determine the direct response of nitrogen molecular sensor to nitrogen, the transformant harboring nitrogen molecular sensor was grown for 5 days under nitrogen-sufficient condition or nitrogen-deficient condition, then the luminescence activity was examined (FIGS. 6 and 7). It was consequently found that the transgenic rice harboring proUPS1::UPS1-LUC2 exhibits a strong luminescence activity in a nitrogen-sufficient condition, indicating the nitrogen sufficiency. While a low luminescence activity was exhibited in a nitrogen-deficient condition, indicating the nitrogen deficiency (FIG. 6). When the transgenic plant is grown for 4 days in a nitrogen-insufficient state for 4 days and then grown for just 1 day under a nitrogen-rich condition, the change in internal nitrogen content in the plant is sensitively recognized by the biological sensor. On the other hand, the transgenic rice harboring proALN::ALN-LUC2 exhibited a low luminescence activity in a nitrogen-sufficient condition, indicating the nitrogen sufficiency in a plant, while a high luminescence activity is exhibited in a nitrogen-deficient condition, indicating the nitrogen deficiency in the plant (FIG. 7).

### Example 7. Nitrogen Specificity of Nitrogen Molecular Sensor Responding to Various Nitrogen Sources

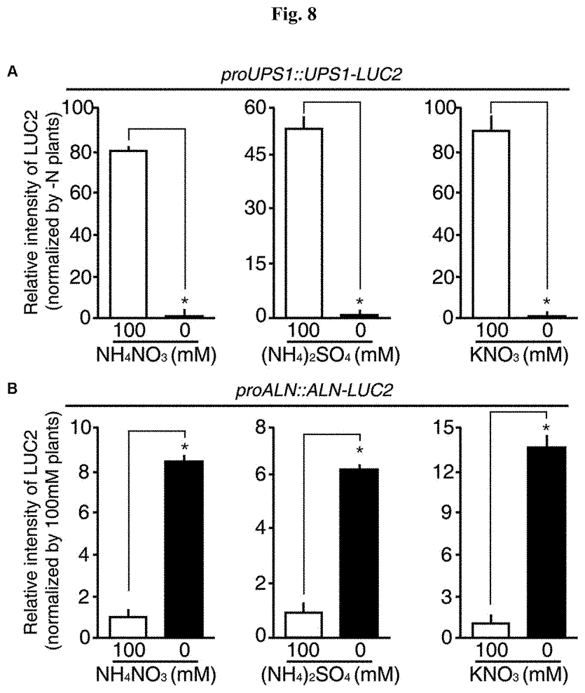

In a transformant harboring nitrogen molecular sensor, the nitrogen molecular sensor exhibits a different luminescence activity sensitive to the nitrogen state, and thus the nitrogen state of a plant can be monitored. In the above Examples, ammonium nitrate was used as a nitrogen source, and thus both ammonia and nitrate, which are common nitrogen sources, are used herein. To determine the nitrogen source specificity of a nitrogen molecular sensor, different nitrogen source, ammonia or nitrate, were supplied separately as a sole nitrogen source, and the response-ability of the nitrogen molecular sensor was examined (FIG. 8). Each nitrogen molecular sensor sensitively exhibited a different luminescence intensity response to different nitrogen source. Thus it allows the monitoring of a nitrogen state of a plant. These results indicate that nitrogen molecular sensors, proUPS1::UPS1-LUC2 and proALN::ALN-LUC2, can sensitively respond to all types of nitrogen as a sensor, and which is valuable in terms of a wide variety of applications.

### Example 8. Response Sensitivity of Nitrogen Molecular Sensor to Nitrogen Source

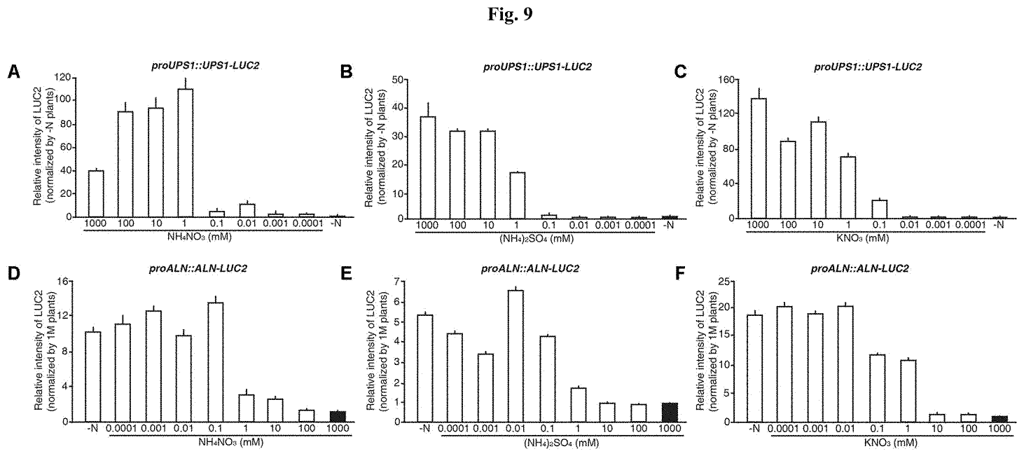

To determine the selectivity of a nitrogen molecular sensor to a different nitrogen source, various concentrations of ammonia or nitrate were separately used as a sole nitrogen source, and the response of a nitrogen molecular sensor was monitored (FIG. 9). The transgenic rice plant, harboring proUPS1::UPS1-LUC2, exhibited a strong luminescence activity in 1 mM or higher concentration of the nitrogen source. In comparison, it showed a low luminescence activity in 1 mM or lower concentration of nitrogen source. While, the transgenic rice plant, harboring proALN::ALN-LUC2, exhibited a low luminescence activity in 1 mM or higher concentration of the nitrogen source. In comparison, it showed a high luminescence activity in 1 mM or lower concentration of nitrogen source. These results suggest that, following the luminescence activity, the nitrogen molecular sensor can be used as a system for non-destructive monitoring of the nitrogen state in a living organism.

1. A nitrogen molecular sensor for detecting nitrogen content in a plant, the nitrogen sensor comprising a plant transformed with at least one of (i) a first expression vector including ALN (ALLANTOINASE) gene derived from Oryza sativa and a gene encoding a luminescent protein and (ii) a second expression vector including UPS1 (UREIDE PERMEASE 1) gene derived from the Oryza sativa and the gene encoding the luminescent protein.

2. The nitrogen molecular sensor of claim 1, wherein the ALN gene consists of the nucleotide sequence of SEQ ID NO: 1 and the UPS1 gene consists of the nucleotide sequence of SEQ ID NO: 2.

3. The nitrogen molecular sensor of claim 1, wherein the luminescent protein is selected from the group consisting of luciferase, green fluorescent protein (GFP), enhanced green fluorescent protein (EGFP), cycle 3 variant of GFP (GFPuv), enhanced blue fluorescent protein (EBFP), enhanced cyan fluorescent protein (ECFP), yellow fluorescent protein (YFP), and a combination thereof.

4. A method for measuring nitrogen content in a plant, the method comprising:
preparing a transgenic plant by transforming a plant with at least one of (i) a first expression vector including ALN (ALLANTOINASE) gene derived from Oryza sativa and a gene encoding a luminescent protein and (ii) a second expression vector including UPS1 (UREIDE PERMEASE 1) gene derived from the Oryza sativa and the gene encoding the luminescent protein; and
cultivating the transgenic plant and measuring luminescence intensity of the transgenic plant.

5. The method according to claim 4, wherein the ALN gene consists of the nucleotide sequence of SEQ ID NO: 1 and the UPS1 gene consists of the nucleotide sequence of SEQ ID NO: 2.

6. The method according to claim 4, wherein the luminescent protein is selected from the group consisting of luciferase, green fluorescent protein (GFP), enhanced green fluorescent protein (EGFP), cycle 3 variant of GFP (GFPuv), enhanced blue fluorescent protein (EBFP), enhanced cyan fluorescent protein (ECFP), yellow fluorescent protein (YFP), and a combination thereof.

7. A composition for measuring nitrogen content in a plant, the composition comprising as an effective component at least one of a first expression vector including ALN (ALLANTOINASE) gene derived from Oryza sativa and a gene encoding a luminescent protein and (ii) a second expression vector including UPS1 (UREIDE PERMEASE 1) gene derived from the Oryza sativa and the gene encoding the luminescent protein.

8. The composition of claim 7, wherein the composition comprises the first expression vector.

9. The composition of claim 8, wherein the ALN gene consists of the nucleotide sequence of SEQ ID NO: 1.

10. The composition of claim 7, wherein the composition comprises the second expression vector.

11. The composition of claim 10, wherein the UPS1 gene consists of the nucleotide sequence of SEQ ID NO: 2.

12. The composition of claim 7, wherein the luminescent protein is selected from the group consisting of luciferase, green fluorescent protein (GFP), enhanced green fluorescent protein (EGFP), cycle 3 variant of GFP (GFPuv), enhanced blue fluorescent protein (EBFP), enhanced cyan fluorescent protein (ECFP), yellow fluorescent protein (YFP), and a combination thereof.

13. The nitrogen molecular sensor of claim 1, wherein the plant is transformed with the first expression vector.

14. The nitrogen molecular sensor of claim 13, wherein the ALN gene consists of the nucleotide sequence of SEQ ID NO: 1.

15. The nitrogen molecular sensor of claim 1, wherein the plant is transformed with the second expression vector.

16. The nitrogen molecular sensor of claim 15, wherein the UPS1 gene consists of the nucleotide sequence of SEQ ID NO: 2.

17. The method of claim 4, wherein the transgenic plant is prepared by transforming the plant with the first expression vector.

18. The method of claim 17, wherein the ALN gene consists of the nucleotide sequence of SEQ ID NO: 1.

19. The method of claim 4, wherein the transgenic plant is prepared by transforming the plant with the second expression vector.

20. The method of claim 19, wherein the UPS1 gene consists of the nucleotide sequence of SEQ ID NO: 2.


SEQUENCE LISTING (2 sequences)

SEQ ID NO: 1 - DNA, 3730 bp, Oryza sativa
  misc_feature (174)..(175): n is a, c, g, or t
  misc_feature (179)..(180): n is a, c, g, or t
  misc_feature (186)..(187): n is a, c, g, or t
  misc_feature (193)..(193): n is a, c, g, or t
agctcaagctaagcttcgtccactcgccggagaacatctccgccgcctcacctcccttccttgtcgacgccaactgctccggagctcgcgcggtcgggggcgggggtggtcgaggagggcggaggcgtcggcgtggcgccggtgagggtggaggcggagacgagggagacaagnngggnngggggnngggggnagagagggaggaaagaggaagtggaccgccgcgcggcgttttcttcggcttgcgctccgtttcttcttttccaacggggggacccaaacgggagctatggtaatttctcgcaaaggcccttgcagaatttggtaatttttcagggtaaataaggaaatggtactgtatactattggaaagctcaaagccattccatttcagaaacgggaacgggaacggggacggaaatgctatggtaatttctcgcaaaggcccttgcagaatttggtaatttttcagggtaaataaggaaatggtactgtatactattggaaagctcaaagccattccatttcagaaaaaaataaatttccatggttctggtatcccacttctaacatagtttgagagttcatgctagtcacgttgctactattatgtgcaattgtgctttcaacgtggcttaaatttatctactggtggctgatgtcatttcttaaagccatggtgatactaataataaattgctactctctccgttttgaaatatatgacatcattgactcttcgcataatgtttgatcatatatcttagttgaaaaatgttatatgcatatcatttattttgttgtaatttttaatcaaccgaggagttattactaaaattttgaataaaacaaataatcgaacgttatataaaaaatcaacatattttgtgatggagatagtacatacgagactcggataagcaccataatactacagtcccatcacaatattcaaagctggagtttactggaggcatggaaatataaaatgattttcagctctatgtacacacgtctgtatgcgctatataaagatgagagtaaattacaaaataaaacggtatcccaccaaattataaaaagaaaatgtatgataagccactaacaaatactacctccatcccaaaatgtttgacgctgttggctttttaaaaaatgtttgaccgttcgtcttattcaaaaaatttaagtaattgttaaatctttttctatcatttgatttattgttaaatatacttttatatatacatatagttttacacatatcacaaaagtttttgaataaaacgaacggtcaaacatgttttaaaaagtcaacggtgtcaaacatttagggaaggagggagtaccaaatgttgactacttgtcaaattagccgctggtttcctcttatacagctggaaagaattttatgtcctaccccacgtagaattttccacctatagcgcaggccgctataagagcaggtacaatagcaggctataagccagctgtaaacatattttaataagataaaagatgaaagagatgagcaacgggctacagatctatagccagctgcagcacggattccaagatgtaatgtgtgtatgacatgtgagaccatatattaatagtatagtaagcaactattgtataaattgtctattagatcggctatagatgaattggagccagtagtgagctatactattataaacttgctctaataattgaataatttgatttgttttatttaaatcttaatttcagttcctatccagtgacgtttcaaaccccgagcaggcaatccacgccgtccatctccgcggccgcgtcacacccaccccctcaccgctacaacgagattctctaattccttaaaacaacacacgagattctctaatttattaataaaaaaataaaaaagaatctcgattttcttataaaaaaacgaaaagaaagactaaaaggcaagagatccctccaaaggttccagccacttggatgccctcacacaacgcaacaaaaccagaatcggatcatccaggtcgcggtccactagaccacgccacagacaggtgggccccacaggaatcctcgtacccaccagtccactggaccaggtgtacagatccactcccacccgactcagatacccgaatccacgtcagcccagagccagtggaggcgcagcccacgcgcacgcgactcgagatggcgatggcggcggcgaagggcagggtcctgccgctgctggcggtggcggcggcgctcgcggcggcgctcctctaccgcgcgcccttttccaagagcctgggcggtgaggggtgcagccttctcccccacgaccacttctggatcgccagcgagcgcgtcgtcacgctcggacgggtcggccccgccgcagtggaggtgaagggagggctgatcaacgcgatcgccgtcggggattaccggagcttcctgctccggcgaccggtggtggactacggtgacgcggttatcatgcccggactgatcgatgtgcacgcgcatcttgatgagccaggacgtgctgagtgggaggggttctccactggtacaagagcagcagctgcaggtggcattacaacattagtggacatgcctcttaatagctacccgtcaactgtttctgaagaaactctcaagctgaagttggatgcagctaaggataaactacatgttgatgtagggttctggggaggcctcgttccagagaatgccctcaatccaagtgcactagagagcctattaaatgcaggcgtcttagggctcaagtcatttatgtgcccctcaggtataaatgacttccccatgacaaattcaactcatattgaggagggcctggttacattggcaaagtacaaaaggccgttacttatccatgcagaacgcatacccgatgttcagaatgaagatgggatcgatggtgaactagatcctaaagcctatacgacatatcttaaatctagaccaccagcatgggaggaagcagcgatcaaagatctgcaacgtgcgatgaaagacacggagatagggggtcggtcagaaggagctcatattcacatagttcatctctcagatgcaaaaacttctcttgggcttcttaaggatgcaaaacaaaatggtgcaagggttagtgtagaaacctgtcctcattacctggcgttttcagctgaagaagttcctgatggagatactcgctttaaatgtgctcctcctatacgcgattcaaccaacagagataatctctgggaagctctgcttgatggacacatagacatgttgagctcagaccattcaccatcagctcctgatctcaaactcatggaggaaggtaacttcttaagggcatggggaggcatatcatctttgcagtttgttctccctgtaacatggtcgcatgggaaaaaatacggtatcagtttgaatcaactggcatcatggtggagcgaaaggcctgctatgcttgcagggttaaagaaaaagggtgctgttctgccgggataccgtgctgatattgtggtatggaaacctgaggctcaattccatcttgatgacagccatcctgtctaccataagcatcggaatatttcggcatatttgggaaagcagctttcagggaagatactgtccacctttgtgggtgggaatcttgtgttcgctgaagacaaacatgcaaaggctgcatgtggtgctccaatcctcgccaaactcgagatggaag

SEQ ID NO: 2 - DNA, 3240 bp, Oryza sativa
agctcaagctaagcttattcggttgcattgcggtgctgccggcgcagacccgactaggcgtaaatataaagaaaagagaaaatatcatttggtgaaatggtgacgagttccagataataatgaactgattcatcttaggccttgtttagttcgtgaaaataaaatttttaggtgtcacatcgaatatttgactgaatgtcggaaggggttttcggacacgaatgaaaaaactaatttcataactcacctagaaaccgcgagactaatcttttgagcctaattaagtcgtcattagcacatgtgggttactgtagtacttatggataatcatggactaattaggctcaaaagactcgtctcgagatttctatacaaactgtgcaattagtttttgtttttatctatgtttaatgcttcatatatgtgcacaaaaattcgatgtgatgttttttgagctaaaccaggccttactactgctactactgcaaatgtgcaatgcacctcaaggagggcagtttagatctgacaaagaggataaaatttctgcaattattggggtacggtccacctaaggtgtgggcccaacagatcacatcctagtcacccggttggtgaactggctcgtgtgggccccacctgctcccaatctccattctcctcatcacatatccataagtctgctactactacttaattagctaaggtttagctatagctaagctcactgactatgcatgaagcttccagaggcagtgaggcaccacaaattttcagacacatgctggctggagtagtgtaaatatacacacacacacactctctctctcatagtaacagggctcaagggccaacaagattagcactcactggttcttcttccttgtttatttggtatgttcattagttctttgctttgcatgaaaaaaagctagctgttatgttgctatttctgcacttaccttgagcaaaactgttgaggtgttttgctgtgaatgtgtgttctgggtgttttggtttgttccaagataatttaacaaggctgctagatctgtttcaagttcagttcttgagctcagagatggtttcgttgtgcttcttgaatctttagcaatcctagagctgtggtctgtggtatgcaggaacatgaagcttctcttctccatttcttccagagtttatgggaatcgttgtggttcacatgttcttggtgttagaacatgcaagtgcatgtgcattgacttcttaatttgttgttcctgcaagtgcatgtgttttctgttatttgctcacacagtgtgttggatcatgttgacttgaattatttatagaagagattgttttggagagcatagatctttggaaagtaatgtccatggattatgctctgcatctttctgcgtcttctttctgatgatgctgaaaatgcttcttgtaatgtttccactccaatccaaagctgttcttagcttcactcttcagtgttccttccttctgtgagttcacttatatggtacagctgaacagagactgatagaagcttggttcctggattgaattttaagttattttagccttttgttgtagaggaatctgatctgtatatgttgtcggaaagttcaggtgaacaattcttctttgttactacctctgctgtttcctgaacagttcttgagctgcaaccatgtagctgaagctgtatacttgtatttcgctttcagagatcacatatttccatcgcacacctctcttacatcttccagagttcagtctatgttttttttttgttctcatgttcttggtttcaatgcaatacacaaaatcctaatgaatctttcccactgagagtttggattgaatcttgttctttccagttcatagctttacacagtgatctgttgctcactgactttgcagagtaacaccgtttaccaagtctgacagagtttcatggagcaggtaattactctgtccctgattcttctccggctccggcgagatcaagcttatgtatcttgtgaaggatatcggcggcgccattgcgctgatgggcgtgtcactggtgctcctgggcacatggccggtggtcctcgccgtgctggagcgccgcggccgcctcccgcagcacacctacctggactactccatcaccaacttcctcgccgccctcctcgtcgccttcaccttcggccagatcggcgccgacacgccggagacgcctaatttcctcacacagctcacccagatgcaggacaactggccttcaatcttgttcgcgatgtcgggtggcatctttaacggcctagggacactggccacgcagtacgggtgggcgttcgtcgggctttcggtcaccgaggtcatggcgtcaagcctgaaggttgtcatagggacaacactgaactattttctcgatggccggatcaacaaggcggagattcttttccccggtgtcggatgctttctgattgcagcctgccttggatctcttgttcactcttccaatgctgctgacaaccaggagaagctgtctagtgccatggtgaaccacagcaacaccaatggggacactgaaaatgaagaactcaccaagcacctccttgagatagaacaacccaaggattgtgaagaacaagagcctgaagtggcaccggctgcccaggctgtggaaaaactagaggcaggaacagctgatttcctggttgaccttgaggggaagagatctattaaagtgctcggatcgaacaccttcctgggcctcggcatcgtcaccttcgccggcgtgagctactccctcttctcgccggcgttcaacctcgccaccaacgaccagtggcacacgctcggggagggcgtcccccacatggtggtgtacacggcattcttctacttctccctcgcctgcttcgccatcggcatcgccctcaacatctggttcctctaccaccccatggtcggcgtgccgcggtcgtcgctcacggcctacctgaaggattggaaggggagggatcttgctctcgtcgcaggcatggtgtgtggccttggcaatgcgttccagttcatggctgggcaggctgctgggtatgctgctgctgacgctgtccaggcgttgccgctggtgagcacggtgtgggggatcgtgctgttcggcgagtaccggagatcgtcgcggcggacttacactctcctggggagcatgctgttcatgttcgtcgtcgccatggtcatcctcatggcgtcgtc